(19) United States
(12) Patent Application Publication
(10) Pub. No.: US 2024/0350397 A1
(43) Pub. Date: Oct. 24, 2024

(54) METHOD FOR PRODUCING FILM

(71) Applicant: KAO CORPORATION, Tokyo (JP)

(72) Inventors: Ryota NAKAJIMA, Odawara-shi (JP); Soyoung PARK, Odawara-shi (JP); Nobuyuki ASAMI, Odawara-shi (JP); Satoshi OZAWA, Wakayama-shi (JP)

(73) Assignee: KAO CORPORATION, Tokyo (JP)

(21) Appl. No.: 18/685,784

(22) PCT Filed: Aug. 25, 2022
(86) PCT No.: PCT/JP2022/032072

§ 371 Date: Feb. 22, 2024

(30) Foreign Application Priority Data
Aug. 26, 2021 (JP) 2021-138129

(51) Int. Cl. A61K 8/81; A61K 8/04; A61K 8/34; A61K 8/86; A61Q 19/00

(52) U.S. Cl. CPC A61K 8/8135; A61K 8/046; A61K 8/34; A61K 8/86; A61Q 19/00; A61K 2800/10; A61K 2800/95

(57) ABSTRACT

A coating-forming composition for forming a coating composed of a fiber-containing deposit directly on skin via electrostatic spraying, the coating-forming composition containing the following component (A), component (B), and component (C): (A) one or more volatile substances selected from the group consisting of water, alcohols, and ketones, (B) a water-insoluble polymer having fiber-forming ability, and (C) one or more polyols. Where (B1) a polymer with a molecular weight of 1×10⁵ or more is contained as the component (B), and a mass content ratio of the component (B1) to the component (B), ((B1)/(B)), is 0.5 or more, and a mass content ratio of the component (B) to the component (C), ((B)/(C)), is 1 or more.

### FIELD OF THE INVENTION

The present invention relates to a method for producing a coating.

### BACKGROUND OF THE INVENTION

Various methods are known to form a coating on the skin or the like via electrostatic spraying. For example, Patent Literatures 1 to 3 describe methods for forming a coating on the skin by electrostatically spraying a composition on the skin. The compositions used in these methods contain a volatile substance, a polymer having coating-forming ability, or the like. By electrostatic spraying, the volatile substance is volatilized to form a coating on the skin or the like, composed of the polymer having coating-forming ability or the like.

There are reports of devices for performing such electrostatic spraying are also reported (Patent Literatures 4 and 5).
Patent Literature 1: JP-A-2006-104211
Patent Literature 2: JP-A-2018-177797
Patent Literature 3: JP-A-2018-177803
Patent Literature 4: JP-A-2003-507165
Patent Literature 5: JP-A-2019-210558

### SUMMARY OF THE INVENTION

The present invention provides a coating-forming composition for forming a coating composed of a fiber-containing deposit directly on the skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
   in which a mass content ratio of the component (B1) to the component (B), ((B1)/(B)), is 0.5 or more, and
   a mass content ratio of the component (B) to the component (C), ((B)/(C)), is 1 or more.

The present invention also provides a composition for forming a coating composed of a fiber-containing deposit directly on the skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.
The present invention also provides a method for producing a coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
in which a mass content ratio of the component (B1) to the component (B), ((B1)/(B)), is 0.5 or more, and
a mass content ratio of the component (B) to the component (C), ((B)/(C)), is 1 or more.

The present invention further provides a method for producing a coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.

### BRIEF DESCRIPTION OF DRAWINGS

FIG. 1 is a schematic diagram showing the configuration of an electrostatic spraying device suitably used in the present invention.

FIG. 2 is a schematic diagram showing how the electrostatic spraying method is carried out using an electrostatic spraying device.

### DETAILED DESCRIPTION OF THE INVENTION

As for the coatings obtained by electrostatically spraying the above-described compositions containing the volatile substance, the polymer having coating-forming ability, or the like it found to have problems such as conspicuous pores, tearing of the coatings, and reduced coating removability, in some sites to which the coatings are applied and some people. As a result of studying the sites and people that develop these symptoms, it found that the symptoms are developed by people with a high level of sebum or at sites with a high level of sebum.

Accordingly, the present invention provides a composition which has high sebum resistance and is for forming a coating on the skin or the like via electrostatic spraying, and a method for producing a coating with high sebum resistance.

As a result of studying various compositional features of a composition for spraying used in the electrostatic spraying method, the present inventors found that, by blending a certain amount of a polyol in addition to a polymer having a specific molecular weight distribution and having fiber-forming ability and a volatile substance, the formed coating is not torn even in the presence of a large amount of sebum, and the coating also has good texture. Furthermore, it has also been found that, when peeling off and removing a coating formed on a site with a high level of sebum, the coating can be easily and cleanly removed, thereby completing the present invention.

According to the present invention, even on the surface of skin or the like where sebum is present in a large amount, it is possible to form a coating composed of a fiber-containing deposit, which is uniform, has high adhesiveness and transparency, causes no tear or lift, and has good texture. Furthermore, when peeling off and removing a coating formed on a site with a high level of sebum, the coating can be easily and cleanly removed.

### Embodiments of the Invention

Hereinafter, the present invention will be described based on its preferred embodiments with reference to drawings.

A method for producing a coating of the present invention is a method for producing a coating, in which a coating composed of a fiber-containing deposit is formed on the surface of an object on which the coating is to be formed, such as skin. In embodiments of the present invention, a composition containing predetermined components is applied to an object on which the coating is to be formed, such as skin, to form a coating. As the method for forming a coating, the electrostatic spraying method is employed in the present invention. In the electrostatic spraying method, a positive or negative high voltage is applied to a composition to charge the composition, and the charged composition is sprayed toward an object on which the coating is to be formed, such as skin. The sprayed composition spreads in space while repeatedly miniaturized by Coulomb repulsive force. In the process of or after adhering to the skin, the solvent, a volatile substance, is dried to thereby form a coating composed of a fiber-containing deposit on the surface of the skin. Note that, as the electrostatic spraying method, the electrostatic spinning method is preferred. Further, a composition used for electrostatic spraying may be referred to as “composition for spraying” or “composition for spinning”.

The above-described composition used in the present invention (hereinafter, this composition is also referred to as “composition for spraying”) is in liquid form in the environment where the electrostatic spraying method is carried out. This composition comprises the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols.

Further, in embodiments of the present invention, from the viewpoint of imparting excellent sebum resistance and good texture to the resulting fiber deposit-containing coating, the component (B) is preferably in the following cases (1) to (4).
(1) A case where (B1) a polymer with a molecular weight of 1×10⁵ or more is contained as the component (B), the mass content ratio of the component (B1) to the component (B), ((B1)/(B)), is 0.5 or more, and the mass content ratio of the component (B) to the component (C), ((B)/(C)), is 1 or more.
(2) A case where the component (B) has a number average molecular weight of 4.5×10⁴ or more.
(3) A case where the composition is obtained by adding the component (A), the component (B), and the component (C), and the component (B) has a number average molecular weight of 4.5×10⁴ or more.
(4) A case where the component (B) is a polymer with a molecular weight distribution peak of more than 9.0×10⁴.

Hereinafter, each component will be described.

The volatile substances of the component (A) are substances having volatility in a liquid state. In the composition for spraying, the component (A) is blended for the purpose that the composition for spraying placed in an electric field is sufficiently charged and then dispensed from the nozzle tip toward an object on which the fiber is formed, such as skin, and as the component (A) evaporates, the charge density of the composition for spraying becomes excessive, and while being further miniaturized by Coulomb repulsion, the component (A) further evaporates, and a dried fiber deposit-containing coating is finally formed. For this purpose, the volatile substances preferably have a vapor pressure at 20° C. of 0.01 kPa or more and 106.66 kPa or less, more preferably 0.13 kPa or more and 66.66 kPa or less, further more preferably 0.67 kPa or more and 40.00 kPa or less, and even more preferably 1.33 kPa or more and 40.00 kPa or less.

Among the volatile substances of the component (A), as the alcohols, monovalent chain aliphatic alcohols, monovalent cyclic aliphatic alcohols, and monovalent aromatic alcohols may, for example, be suitably used. Examples of the monovalent chain aliphatic alcohols, monovalent cyclic alcohols, and monovalent aromatic alcohols include C₁-C₆ alcohols, C₄-C₆ cyclic alcohols, and benzyl alcohol and phenylethyl alcohol, respectively. Specific examples thereof include ethanol, isopropyl alcohol, butyl alcohol, phenylethyl alcohol, n-propanol, and n-pentanol. As for these alcohols, one or more selected from the group consisting of them can be used. Among the volatile substances of the component (A), the alcohols do not include polyols, which are polyhydric alcohols, but are alcohols other than the component (C), which will be described later.

Among the volatile substances of the component (A), examples of the ketones include di-C₁-C₄ alkyl ketones, such as acetone, methyl ethyl ketone, and methyl isobutyl ketone. One of these ketones may be used singly, or two or more of them may be used in combination.

Water can also be contained in the component (A). In the case where water is contained, it is preferable to use water in combination with an alcohol and/or a ketone. The content of water in the component (A) is preferably 0.01% by mass or more and 5% by mass or less from the viewpoint of volatility of the component (A) and adhesiveness of the coating to the skin.

The volatile substances of the component (A) are more preferably one or more selected from the group consisting of ethanol, isopropyl alcohol, butyl alcohol, and water, more preferably one or more selected from the group consisting of ethanol and butyl alcohol, and further more preferably volatile substances containing ethanol.

The content of the component (A) in the composition for spraying is preferably 45% by mass or more, further more preferably 50% by mass or more, and even more preferably 55% by mass or more. Also, it is preferably 95% by mass or less, further more preferably 94% by mass or less, and even more preferably 93% by mass or less. The content of the component (A) in the composition for spraying is preferably 45% by mass or more and 95% by mass or less, further more preferably 508 by mass or more and 94% by mass or less, and even more preferably 55% by mass or more and 93% by mass or less. When the component (A) is contained in the composition for spraying at this proportion, the composition for spraying can be sufficiently volatilized when carrying out the electrostatic spraying method even in the presence of the component (C).

Further, ethanol is preferably 50% by mass or more, further more preferably 65% by mass or more, and even more preferably 80% by mass or more, with respect to the entire amount of the volatile substances of the component (A). It is also preferably 100% by mass or less. Ethanol is preferably 50% by mass or more and 100% by mass or less, further more preferably 65% by mass or more and 100% by mass or less, and even more preferably 80% by mass or more and 100% by mass or less, with respect to the entire amount of the volatile substances of the component (A).

The water-insoluble polymer having fiber-forming ability as the component (B) is generally a substance which can be dissolved in the volatile substances of the component (A). Here, being dissolved refers to being in a dispersed state at 20° C. and that dispersed state is in a visually uniform state, preferably in a visually transparent or semi-transparent state.

As the water-insoluble polymer having fiber-forming ability, those appropriate depending on the nature of the volatile substances of the component (A) are used. In the present specification, “water-insoluble polymer” refers to those having the property that, after weighing 1 g of a polymer and then immersing it in 10 g of ion exchanged water under an environment of 1 atm and 23° C., more than 0.5 g of the immersed polymer is not dissolved after a lapse of 24 hours.

Examples of the water-insoluble polymer having fiber-forming ability (B) include completely saponified polyvinyl alcohols, which can be subjected to insolubilization treatment after fiber formation, partially saponified polyvinyl alcohols, which can be subjected to crosslinking treatment after fiber formation when used in combination with a crosslinking agent, low saponified polyvinyl alcohols, oxazoline-modified silicones such as poly(N-propanoylethyleneimine) graft-dimethylsiloxane/γ-aminopropyl methylsiloxane copolymers, polyvinyl acetal diethylaminoacetate, zein (the main component of corn protein), polyesters, polylactic acid (PLA), polyacrylonitrile resins, acrylic resins such as polymethacrylic acid resins, polystyrene resins, polyvinyl butyral resins, polyethylene terephthalate resins, polybutylene terephthalate resins, polyurethane resins, polyamide resins, polyimide resins, and polyamide-imide resins. One of these water-insoluble polymers may be used singly, or two or more of them may be used in combination.

Among these water-insoluble polymers, it is preferable to use one or more selected from the group consisting of completely saponified polyvinyl alcohols, partially saponified polyvinyl alcohols, low saponified polyvinyl alcohols, polyvinyl butyral resins, polyurethane resins, polymethacrylic acid resins, oxazoline-modified silicones, polyvinyl acetal diethylaminoacetate, and polylactic acid, it is more preferable to use one or more selected from the group consisting of polyvinyl butyral resins, polylactic acid, and polyurethane resins, it is further more preferable to contain a polyvinyl butyral resin, and it is particularly preferable to contain a polyvinyl butyral resin as the main component.

In the present invention, it is preferable that these components (B) have a specific molecular weight or molecular weight distribution from the viewpoint of imparting excellent sebum resistance to the fiber deposit-containing coating. Further, when the component (B) has such a specific molecular weight or molecular weight distribution, the peelability of the sebum-containing coating becomes much better.

The molecular weight of the component (B) is determined by preparing a calibration curve from standard polystyrene using the gel permeation chromatography (GPC) method.

First of all, in one aspect, it is preferable that (B1) a polymer with a molecular weight of 1×10⁵ or more is contained as the component (B), and the mass content ratio of the component (B1) to the component (B), ((B1)/(B)), is 0.5 or more. That is, it is preferable for the component (B) to contain 50% by mass or more of (B1) the polymer with a molecular weight of 1×10⁵ or more.

Further, the mass content ratio of the component (B1) to the component (B), ((B1)/(B)), is preferably 0.51 or more, more preferably 0.53 or more, and further more preferably 0.55 or more, from the viewpoint of imparting excellent sebum resistance to the fiber deposit-containing coating, the viewpoint of improving the texture of the coating, and the viewpoint of peelability of the sebum-containing coating. Here, the upper limit of (B1)/(B) is preferably 1 or less, more preferably 0.95 or less, and further more preferably 0.9 or less, from the same viewpoints.

Next, in another embodiment, it is preferable that the component (B) has a number average molecular weight of 4.5×10⁴ or more. The number average molecular weight of the component (B) is preferably 4.6×10⁴ or more, more preferably 4.7×10⁴ or more, and further more preferably 4.8×10⁴ or more, from the viewpoint of imparting excellent sebum resistance to the fiber deposit-containing coating, the viewpoint of improving the texture of the coating, and the viewpoint of peelability of the sebum-containing coating. Also, the upper limit of the number average molecular weight of the component (B) is preferably 3×10⁵ or less, more preferably 2×10⁵ or less, and further more preferably 1.7×10⁵ or less, from the same viewpoints.

Next, in another embodiment, it is preferable that the composition is obtained by adding the component (A), the component (B), and the component (C), and the component (B) has a number average molecular weight of 4.5×10⁴ or more. The number average molecular weight of the component (B) in the composition obtained by adding the component (B) is preferably 4.6×10⁴ or more, more preferably 4.7×10⁴ or more, and further more preferably 4.8×10⁴ or more, from the viewpoint of imparting excellent sebum resistance to the fiber deposit-containing coating, the viewpoint of improving the texture of the coating, and the viewpoint of peelability of the sebum-containing coating. Also, the upper limit of the number average molecular weight of the component (B) is preferably 3×10⁵ or less, more preferably 2×10⁵ or less, and further more preferably 1.7×10⁵ or less, from the same viewpoints.

Next, in a further embodiment, it is preferable that the component (B) is a polymer with a molecular weight distribution peak of more than 9.0×10⁴. The molecular weight distribution peak of the component (B) is preferably 9.5×10⁴ or more, more preferably 1.0×10⁵ or more, and further more preferably 1.1×10⁵ or more, from the viewpoint of imparting excellent sebum resistance to the fiber deposit-containing coating, the viewpoint of improving the texture of the coating, and the viewpoint of peelability of the sebum-containing coating. Also, the upper limit of the molecular weight distribution peak of the component (B) is preferably 1.0×10⁶ or less, more preferably 7.0×10⁵ or less, further more preferably 5.0×10⁵ or less, and even more preferably 3.5×10⁵ or less, from the same viewpoints.

In a case where the component (B) is a polymer of which main chain functional groups are modified, such as polyvinyl butyral resin, the sebum resistance tends to deteriorate when the modification rate of such functional groups is too high, and therefore, it is preferable that the modification rate of such functional groups is 75% or less. For example, in the case of polyvinyl butyral resin, the acetalization rate (butyralization rate) is preferably 75% or less.

The content of the component (B) in the composition for spraying is preferably 28 by mass or more, further more preferably 48 by mass or more, and even more preferably 5% by mass or more, from the viewpoint of forming the fiber deposit-containing coating with excellent sebum resistance on an object on which the coating is to be formed, such as skin, and the viewpoint of peelability of the sebum-containing coating. Also, it is preferably 30% by mass or less, further more preferably 20% by mass or less, and even more preferably 15% by mass or less. The content of the component (B) in the composition for spraying is preferably 28 by mass or more and 30% by mass or less, further more preferably 4% by mass or more and 20% by mass or less, and even more preferably 5% by mass or more and 15% by mass or less. When the component (B) is contained in the composition for spraying at this proportion, it is possible to efficiently form the target fiber deposit-containing coating.

The mass content ratio of the component (B) to the component (A), ((B)/(A)), in the composition for spraying is preferably 0.03 or more and 0.5 or less, more preferably 0.04 or more and 0.4 or less, and further more preferably 0.055 or more and 0.3 or less, from the viewpoint that the component (A) can be sufficiently volatilized when carrying out the electrostatic spraying method and the target coating can be formed.

Further, the mass content ratio of ethanol (A) to the component (B), ((B)/(A)), in the composition for spraying is preferably 0.03 or more and 0.5 or less, more preferably 0.04 or more and 0.4 or less, and further more preferably 0.055 or more and 0.3 or less, from the viewpoint that ethanol (A) can be sufficiently volatilized when carrying out the electrostatic spraying method and the target coating can be formed.

The component (C) used in the present invention is one or more selected from the group consisting of polyols. By combining the above-described component (B) having a specific molecular weight or molecular weight distribution with such a component (C), it is possible to impart excellent sebum resistance to the fiber deposit-containing coating, and furthermore, the texture of the coating is improved and the peelability of the sebum-containing coating is improved.

Examples of the polyols of the component (C) include alkylene glycols such as ethylene glycol, propylene glycol, 1,3-propanediol, and 1,3-butanediol; polyalkylene glycols such as diethylene glycol, dipropylene glycol, polyethylene glycol with a molecular weight of 1 000 or less, and polypropylene glycol; glycerins such as glycerin, diglycerin, and triglycerin. Among the above, from the viewpoint that excellent sebum resistance can be imparted to the fiber deposit-containing coating and the texture of the coating is improved, and the viewpoint that the peelability of the sebum-containing coating is improved, ethylene glycol, propylene glycol, 1,3-butanediol, dipropylene glycol, polyethylene glycol with a molecular weight of 1 000 or less, glycerin, and diglycerin are preferred, polyethylene glycol with a molecular weight of 1 000 or less, propylene glycol, 1,3-butanediol, and glycerin are further more preferred, and polyethylene glycol with a molecular weight of 1 000 or less and 1,3-butanediol are even more preferred.

The content of the component (C) in the composition for spraying is preferably 0.18 by mass or more, more preferably 0.5% by mass or more, and further more preferably 1% by mass, and also preferably 30% by mass or less, more preferably 25% by mass or less, and further more preferably 20% by mass or less, from the viewpoint that excellent sebum resistance can be imparted to the fiber deposit-containing coating and the texture of the coating is improved, and the viewpoint that the peelability of the sebum-containing coating is improved. Specifically, it is preferably 0.1% by mass or more and 30% by mass or less, more preferably 0.58 by mass or more and 25% by mass or less, and further more preferably 18 by mass or more and 20% by mass or less.

The fiber formed by the electrostatic spraying method, preferably by electrostatic spinning method, from the composition for spraying of the present invention can form a coating as a fiber deposit with the component (B) as the major component and the component (C) being present. The component (C) being present refers to the state in which it is supported inside the fiber, on the fiber surface side, or between the fibers.

In the composition for spraying of the present invention, from the viewpoint of imparting excellent sebum resistance to the fiber deposit-containing coating to improve the texture of the coating and the viewpoint of improving peelability of the sebum-containing coating, the mass content ratio of the component (B) to the component (C), ((B)/(C)), is preferably 1 or more. Such a ratio can impart excellent sebum resistance to the fiber deposit-containing coating, improve the texture of the coating, and improve the peelability of the sebum-containing coating.

The ((B)/(C)) is more preferably 1.1 or more, and further more preferably 1.2 or more, from the same viewpoints. Further, the upper limit of the ((B)/(C)) is preferably 30 or less, more preferably 25 or less, and further more preferably 20 or less, from the same viewpoints.

Specifically, it is preferably 1 or more and 30 or less, more preferably 1.1 or more and 25 or less, and further more preferably 1.2 or more and 20 or less.

The composition for spraying of the present invention may further comprise a component (D) oil. The (D) oil is an oil which is in liquid form (liquid form at 20° C.) as a whole, and may contain a semi-solid oil or a solid oil in addition to an oil which is liquid at 20° C. (oil in liquid form).

Examples of the oil in liquid form include hydrocarbon oils, ester oils, higher alcohols, silicone oils, and fatty acids. Among the above, hydrocarbon oils, ester oils, and silicone oils are preferred from the viewpoint that excellent sebum resistance can be imparted to the fiber deposit-containing coating and the texture of the coating is improved. Further, one or more selected from the group consisting of these oils in liquid form can be used in combination.

Examples of the hydrocarbon oils in liquid form include liquid paraffin, squalane, squalene, n-octane, n-heptane, cyclohexane, light isoparaffin, liquid isoparaffin, hydrogenated polyisobutene, polybutene, and polyisobutene, and from the viewpoint that excellent sebum resistance can be imparted to the fiber deposit-containing coating and the texture of the coating is improved, liquid paraffin, light isoparaffin, liquid isoparaffin, squalane, squalene, n-octane, n-heptane, and cyclohexane are preferred, and liquid paraffin and squalane are more preferred.

Further, from the viewpoint that excellent sebum resistance can be imparted to the fiber deposit-containing coating and the texture of the coating is improved, the viscosity at 30° C. of the hydrocarbon oils is preferably 1 mPa·s or more, and more preferably 3 mPa·s or more. Further, from the viewpoint that excellent sebum resistance can be imparted to the fiber deposit-containing coating and the texture of the coating is improved, the total content of isododecane, isohexadecane, and hydrogenated polyisobutene in the liquid agent is preferably 10% by mass or less, more preferably 5% by mass or less, further more preferably 18 by mass or less, and even more preferably 0.5% by mass or less, and they may not be contained.

Similarly, from the viewpoint that excellent sebum resistance can be imparted to the fiber deposit-containing coating and the texture of the coating is improved, the viscosity at 30° C. of the ester oils and silicone oils is preferably 1 mPa·s or more, and more preferably 3 mPa·s or more.

The viscosity here is measured at 30° C. with a BM-type viscometer (manufactured by Tokimec, Inc., measurement conditions: rotor No. 1, 60 rpm, 1 minute). Note that, from the same viewpoint, the total content of ether oils such as cetyl-1,3-dimethylbutyl ether, dicaprylyl ether, dilauryl ether, and diisostearyl ether in the component (D) is preferably 10% by mass or less, more preferably 58 by mass or less, and further more preferably 1% by mass or less.

Examples of the ester oils include esters composed of linear or branched fatty acids and linear or branched alcohols or polyhydric alcohols. Specific examples thereof include isopropyl myristate, cetyl isooctanoate, isocetyl octanoate, octyldodecyl myristate, isopropyl palmitate, butyl stearate, hexyl laurate, decyl oleate, octyldodecyl oleate, hexyldecyl dimethyloctanoate, cetyl lactate, myristyl lactate, lanolin acetate, isocetyl stearate, isocetyl isostearate, ethylhexyl isononanoate, isononyl isononanoate, isotridecyl isononanoate, isostearyl isostearate, cholesteryl 12-hydroxystearate, ethylene glycol di-2-ethylhexanoate, dipentaerythritol fatty acid ester, n-alkyl glycol monoisostearate, propylene glycol dicaprylate, propylene glycol diisostearate, neopentyl glycol dicaprate, diisostearyl malate, glycerin di-2-heptylundecanoate, trimethylolpropane tri-2-ethylhexanoate, trimethylolpropane triisostearate, pentaerythrite tetra-2-ethylhexanoate, glyceryl tri-2-ethylhexanoate, trimethylolpropane triisostearate, cetyl 2-ethylhexanoate, 2-ethylhexyl palmitate, diethylhexyl naphthalene dicarboxylate, (C12-15 alkyl) benzoate, cetearyl isononanoate, caprylic/capric triglyceride, butylene glycol dicaprylate/dicaprate, propylene glycol dicaprylate/dicaprate, glyceryl triisostearate, glyceryl tri-2-heptylundecanoate, glyceryl tri-coconut oil fatty acid, methyl ester of castor oil fatty acid, oleyl oleate, 2-heptylundecyl palmitate, diisobutyl adipate, 2-octyldodecyl N-lauroyl-L-glutamate ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, di-2-ethylhexyl succinate, triethyl citrate, 2-ethylhexyl para-methoxycinnamate, and tripropylene glycol dipivalate.

Among the above, from the viewpoint that excellent sebum resistance can be imparted to the fiber deposit-containing coating and the texture of the coating is improved, at least one selected from the group consisting of octyldodecyl myristate, myristyl myristate, isocetyl stearate, isononyl isononanoate, isocetyl isostearate, cetearyl isononanoate, diisobutyl adipate, di-2-ethylhexyl sebacate, isopropyl myristate, isopropyl palmitate, diisostearyl malate, neopentyl glycol dicaprate, and caprylic/capric triglyceride is preferred, at least one selected from the group consisting of isopropyl myristate, isopropyl palmitate, diisostearyl malate, neopentyl glycol dicaprate, (C12-15 alkyl) benzoate, and caprylic/capric triglyceride is more preferred, and at least one selected from the group consisting of neopentyl glycol dicaprate and caprylic/capric triglyceride is further more preferred.

Further, as the ester oils, it is possible to use vegetable oils and animal oils including the above-described ester oils, and examples thereof include olive oil, jojoba oil, macadamia nut oil, meadowfoam oil, castor oil, safflower oil, sunflower oil, avocado oil, canola oil, apricot kernel oil, rice germ oil, and rice bran oil.

Examples of the higher alcohols include liquid higher alcohols having 12 to 20 carbon atoms, and higher alcohols with branched fatty acids as the constituent are preferred. Specific examples thereof include isostearyl alcohol and oleyl alcohol.

Examples of the liquid silicone oils include linear silicones, cyclic silicones, and modified silicones, such as dimethylpolysiloxane, dimethylcyclopolysiloxane, methylphenylpolysiloxane, methylhydrogenpolysiloxane, phenyl-modified silicone, and higher alcohol-modified organopolysiloxane.

The component (D) may also contain an oil in solid form at 20° C., as long as the component (D) is in liquid form as a whole. The oil in solid form at 20° C. is not particularly restricted as long as it is normally used in cosmetics. Examples thereof include waxes, cholesterol derivatives, phytosterol derivatives, dipentaerythrite fatty acid esters, triglycerides, lanolin, lanosterol derivatives, vaseline, ceramides, higher alcohols, and higher fatty acids.

The content of the component (D) in the composition for spraying is preferably 08 by mass or more, and preferably 25% by mass or less, more preferably 20% by mass or less, and further more preferably 15% by mass or less, from the viewpoint that excellent sebum resistance can be imparted to the fiber deposit-containing coating and the texture of the coating is improved. Specifically, it is preferably 0% by mass or more and 25% by mass or less, more preferably 0% by mass or more and 20% by mass or less, and further more preferably 0% by mass or more and 15% by mass or less. Note that, from the viewpoint of maintaining transparency of the coating, it is preferable that the content is 0.5% by mass or more.

The composition for spraying may comprise only the above-described component (A), component (B), and component (C), may comprise the component (A), component (B), component (C), and component (D), or may comprise other components in addition to the component (A), component (B), component (C), and component (D). Examples of other components include plasticizers, coloring pigments, extender pigments, dyes, surfactants, flavors, repellents, antioxidants, stabilizers, preservatives, and various vitamins, other than the component (B), polymer having fiber-forming ability. In the case where other components are contained in the composition for spraying, it is preferable that the content of such other components is 0.1% by mass or more and 30% by mass or less, and further more preferably 0.5% by mass or more and 20% by mass or less.

In the method for producing a coating of the present invention, the above-described composition for spraying is electrostatically sprayed onto the surface of an object on which the coating is to be formed, thereby forming a coating composed of a fiber-containing deposit. Here, the object on which the coating is to be formed may be a base material such as resin, glass, or metal, or may be skin, although skin is preferred. In the case where the coating is formed on the skin, the above-described composition for spraying is electrostatically sprayed directly onto the skin surface to thereby form a coating composed of a fiber-containing deposit on the skin surface. In order to efficiently form a fiber-containing deposit, it is preferable to employ the electrostatic spinning method among the electrostatic spraying method.

In the case of carrying out the electrostatic spraying method, the composition for spraying, preferably composition for spinning, to be used is one having a viscosity at 25° C. of preferably 1 mPa·s or more, further more preferably 10 mPa·s or more, and even more preferably 50 mPa·s or more. Also, the composition to be used is one having a viscosity at 25° C. of preferably 5 000 mPa·s or less, further more preferably 2 000 mPa·s or less, and even more preferably 1 500 mPa·s or less. The viscosity of the composition for spraying at 25° C. is preferably 1 mPa·s or more and 5 000 mPa·s or less, further more preferably 10 mPa·s or more and 2 000 mPa·s or less, and even more preferably 50 mPa·s or more and 1 500 mPa·s or less. By using a composition for spraying having a viscosity in this range, a porous coating, in particular porous coating composed of a fiber deposit, can be successfully formed by the electrostatic spraying method. The formation of a porous coating is advantageous from the viewpoints of adhesiveness of the coating, transparency of the coating, suppression of coating feeling, prevention of skin stuffiness, and the like. The viscosity of the composition for spraying is measured at 30° C. using an E-type viscometer. As the E-type viscometer, for example, an E-type viscometer manufactured by Tokyo Keiki Inc. can be used. As a rotor in such a case, Rotor No. 43 may be used.

The composition for spraying is sprayed or dispensed directly on an object on which the coating is to be formed, preferably target site of human skin, by the electrostatic spraying method.

Here, the skin also includes nails. The electrostatic spraying method includes a step of electrostatically spraying the composition for spraying on the skin using an electrostatic spraying device. The electrostatic spraying device basically has a container which accommodates the composition, a nozzle which dispenses the composition, a supplying device which supplies the composition accommodated in the container to the nozzle, and a power source which applies a voltage to the nozzle.

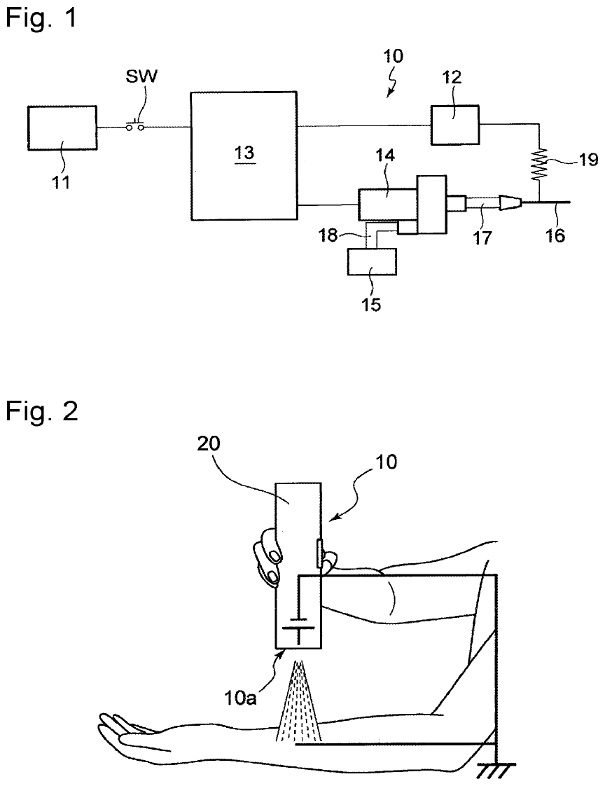

FIG. 1 shows a schematic diagram representing the configuration of an electrostatic spraying device suitably used in the present invention. An electrostatic spraying device 10 shown in the figure comprises a low voltage power source 11. The low voltage power source 11 can generate a voltage of several to a dozen volts. For the purpose of enhancing the portability of the electrostatic spraying device 10, it is preferable that the low voltage power source 11 is composed of one or more batteries. The use of batteries as the low voltage power source 11 also has the advantage that they can be easily replaced as necessary. Instead of batteries, an AC adapter or the like may be used as the low voltage power source 11.

The electrostatic spraying device 10 also comprises a high voltage power source 12. The high voltage power source 12 is connected to the low voltage power source 11, and comprises an electrical circuit (not shown) which boosts the voltage generated by the low voltage power source 11 to a higher voltage. Booster electrical circuits are generally composed of transformers, capacitors, semiconductor elements, and others.

The electrostatic spraying device 10 further comprises an auxiliary electrical circuit 13. The auxiliary electrical circuit 13 intervenes between the above-described low voltage power source 11 and high voltage power source 12, and has the function of adjusting the voltage of the low voltage power source 11 to ensure stable operation of the high voltage power source 12. Furthermore, the auxiliary electrical circuit 13 has the function of controlling the rotation speed of the motor provided in a micro gear pump 14, which will be described later. By controlling the rotation speed of the motor, the amount of the composition for spraying supplied from a container 15, which will be described later, for the composition for spraying to the micro gear pump 14 is controlled. A switch SW is mounted between the auxiliary electrical circuit 13 and the low voltage power source 11, and the electrostatic spraying device 10 can be operated/stopped by turning the switch SW on and off. Note that control of the supply and amount of the composition for spraying supplied can also be performed using a piston pump in addition to the micro gear pump 14.

The electrostatic spraying device 10 further comprises a nozzle 16. The nozzle 16 is composed of various conductive materials, including metals, and non-conductive materials such as plastics, rubbers, and ceramics, and is shaped to allow the composition for spraying to be dispensed from the tip thereof. A minute space in which the composition for spraying is distributed is formed in the nozzle 16 along the longitudinal direction of the nozzle 16. The size of the transverse section of this minute space is preferably 100 μm or more and 1 000 μm or less, expressed as a diameter.

The nozzle 16 is connected to the micro gear pump 14 via a conduit line 17. The conduit line 17 may be composed of a conductive material or a non-conductive material. Also, the nozzle 16 is electrically connected to the high voltage power source 12. This makes it possible to apply a high voltage to the nozzle 16. In this case, in order to prevent an excessive current from flowing when the nozzle 16 is in direct contact with the human body, the nozzle 16 and the high voltage power supply 12 are electrically connected via a current-limiting resistor 19.

The micro gear pump 14, which is connected to the nozzle 16 via the conduit line 17, functions as a supplying device which supplies the composition for spraying accommodated in the container 15 to the nozzle 16. The micro gear pump 14 operates by receiving power supply from the low voltage power source 11. Further, the micro gear pump 14 is configured to supply a predetermined amount of the composition for spraying to the nozzle 16 under the control of the auxiliary electrical circuit 13.

To the micro gear pump 14, the container 15 is connected via a flexible conduit line 18. In the container 15, the composition for spraying is accommodated. The container 15 is preferably in the form of a replaceable cartridge type.

The electrostatic spraying device 10 having the configuration as described above can be used as shown in FIG. 2, for example. FIG. 2 shows a handheld type electrostatic spraying device 10 having dimensions which can be grasped with one hand. As for the electrostatic spraying device 10 shown in the figure, all of the members in the configuration diagram shown in FIG. 1 are accommodated in a cylindrical housing 20. A nozzle (not shown) is arranged at one end 10a of the housing 20 in the longitudinal direction. The nozzle is arranged in the housing 20 such that the direction in which it blows out the composition matches the longitudinal direction of the housing 20 and is convex toward the skin side. By arranging the nozzle tip in the longitudinal direction of the housing 20 such that it is convex toward the skin, the composition for spraying is unlikely to adhere to the housing, and a coating can be formed in a stable manner.

When operating the electrostatic spraying device 10, the user, that is, the person who will form a coating on the target site on the skin by electrostatic spraying, grasps the device 10 by hand and points the one end 10a of the device 10, where the nozzle (not shown) is arranged, to the application site where the electrostatic spraying is to be carried out. FIG. 2 shows the state where the one end 10a of the electrostatic spraying device 10 is pointed toward the inside of the forearm of the user. Under this state, the electrostatic spraying method is carried out by turning on the switch of the device 10. When the device 10 is powered on, an electric field is generated between the nozzle and the skin. In the embodiment shown in FIG. 2, a positive high voltage is applied to the nozzle and the skin becomes the negative electrode. When an electric field is generated between the nozzle and the skin, the composition for spraying at the nozzle tip is polarized by electrostatic induction to make the tip cone-shaped, and droplets of the charged composition for spraying are dispensed from the cone tip into the air along the electric field and toward the skin. As the solvent, component (A), evaporates from the charged composition for spraying dispensed into the space, the charge density on the surface of the composition for spraying becomes excessive, and it spreads in the space while being repeatedly miniaturized by Coulomb repulsive force, and reaches the skin. In this case, by adjusting the viscosity of the composition for spraying as appropriate, the sprayed composition can reach the application site in a droplet state. Alternatively, while being dispensed into the space, the solvent, volatile substance, can be volatilized from the droplets to solidify the solute, polymer having fiber-forming ability, and fibers can be formed while being elongated and deformed by the electric potential difference and the fibers can be deposited on the application site. For example, when the viscosity of the composition for spraying is increased, it is easier to deposit the composition in the form of fibers on the application site. This forms a porous coating composed of a fiber deposit on the surface of the application site.

While carrying out the electrostatic spraying method, a high potential difference is generated between the nozzle and the skin. However, the impedance is so large that the current flowing through the human body is extremely minute. For example, the present inventors have confirmed that the current flowing through the human body while carrying out the electrostatic spraying method is several orders of magnitude smaller than the current flowing through the human body due to static electricity generated under normal conditions of life.

In the case of skin containing a high level of sebum, complexation of the component (C) in the fiber makes it easier for the fiber to be swollen and plasticized. As a result, the resulting coating is transparent and has good adhesiveness, but its durability, that is, sebum resistance, is considered to be decreased. In contrast, the use of a polymer having fiber-forming ability with a specific molecular weight or molecular weight distribution as the component (B) is thought to improve durability in the presence of sebum and prevent tearing or lifting, even though the coating is flexible.

The contents of the component (A), component (B), and component (C), composition for spraying, are measured as follows. Since the volatile substance, component (A), is not present in the formed coating, or volatilizes even if present, the formed coating contains the component (B) and component (C) as the major components, and their contents are measured as follows.

### <Method for Measuring Contents of Component (A), Component (B), and Component (C) in Composition for Spraying>

There are methods such as separation and identification by liquid chromatography (HPLC) in a solution state or identification with an infrared spectrophotometer (IR). Since liquid chromatography allows elution from components with larger molecular weights, the molecular weight can be predicted and the compositional features can also be identified by the elution position of the component. IR analysis allows for the attribution and identification of functional groups from individual absorbers, and in general, identification can be performed by comparing the standard chart of the commercial additive with the IR chart of the component.

### <Method for Measuring Contents of Component (B) and Component (C) in Formed Coating>

Solvents that can dissolve the coating are searched for, and after dissolving the coating in the solvent, separation and identification by liquid chromatography (HPLC), or identification with an infrared spectrophotometer (IR), are performed.

Although the fiber which forms the coating is a continuous fiber with infinite length in principle of production, it is preferable to have a length of at least 100 times the thickness of the fiber or more. In the present specification, a fiber having a length of at least 100 times the thickness of the fiber or more is defined as “continuous fiber”. Then, the coating produced by the electrostatic spraying method is preferably a porous discontinuous coating composed of a continuous fiber deposit. The coating in such a form not only can be handled as a single sheet as an aggregate, but also has the advantage of being characterized as very soft and does not easily come apart when shearing force is applied to it, thus providing excellent followability to body movements. It also has the advantage of easy removal by peeling off the coating. In contrast, continuous coatings without pores are not easy to peel off and have low dissipation of sweat, which may cause stuffiness on the skin. Here, requiring pores refers to pores between fibers, and also includes the case where, for example, a liquid substance is present between fibers. Further, as for porous discontinuous coatings composed of an aggregate of particles, it is difficult to completely remove the coating without damaging the skin, for example, it is necessary to apply friction to the entire coating in order to completely remove the coating.

The thickness of the fiber which forms the coating is preferably 10 nm or more, more preferably 50 nm or more, and further more preferably 100 nm or more, in the case where it is expressed as an equivalent circle diameter. Further, it is preferably 3 000 nm or less, more preferably 1 500 nm or less, and further more preferably 1 200 nm or less. The thickness of the fiber can be measured by, for example, observing the fiber under scanning electron microscopy (SEM) at a magnification of 10 000 times, excluding defects (fiber lumps, fiber crossings, droplets) from the two-dimensional image, arbitrarily selecting 10 fibers, drawing a line orthogonal to the longitudinal direction of the fiber, and directly reading the fiber diameter.

In the electrostatic spraying step using the electrostatic spraying device 10, the electrostatically sprayed composition for spraying, which has become fibrous, reaches the skin directly with the component (B) and component (C) in a charged state, while the component (A) evaporates. As previously described, the skin is also charged, and therefore, the fiber closely adheres to the skin in the form of a single film due to electrostatic force. Since fine irregularities such as skin texture are formed on the skin surface, the fiber is thought to further closely adhere to the skin surface in the form of a single film, coupled with the anchoring effect due to the irregularities. When the electrostatic spraying is completed as described above, the electrostatic spraying device 10 is powered off. This causes the electric field between the nozzle and the skin to disappear, and the charge on the skin surface is fixed. As a result, the adhesiveness of the coating in the form of a single film is further developed, making it difficult for the coating to peel off from the edges during wear and improving durability during use. Further, since the fiber which constitutes the coating contains the component (C), the coating can closely adhere to the skin sufficiently even without separately applying a liquid to the skin. The reasons for this are thought to be that the presence of the component (C) in the fiber makes the fiber itself softer due to the plasticizing effect and increases followability to a fine irregular surface, and that the component (C) bleeds out onto the fiber surface to thereby form liquid crosslinking between the fiber and the skin.

Furthermore, since there is a supporting coating in which the component (C) is present between fibers of the fiber which constitutes the coating or on the fiber surface, the fiber which constitutes the coating is unlikely to reflect light, the appearance of the coating tends to become transparent, and the skin can be covered with appearance in a natural state.

The distance between the nozzle and the skin depends on the voltage applied to the nozzle, but it is preferably 50 mm or more and 150 mm or less for successful formation of the coating. The distance between the nozzle and the skin can be measured with a commonly used non-contact sensor or other means.

Regardless of whether the coating formed by the electrostatic spraying method is porous or not, the basis weight of the coating is preferably 0.1 g/m² or more, and further more preferably 1 g/m² or more. Also, it is preferably 50 g/m² or less, and further more preferably 40 g/m² or less. For example, the basis weight of the coating is preferably 0.1 g/m² or more and 50 g/m² or less, and further more preferably 1 g/m² or more and 40 g/m² or less. By setting the basis weight of the coating as such, the adhesiveness of the coating can be improved.

Note that the electrostatic spraying step in which the composition is electrostatically sprayed directly on the skin to form a coating means a step in which electrostatic spraying is performed on the skin to form a coating, and it is preferably a step in which electrostatic spinning is performed.

Examples of other methods for forming a coating include a method which includes a step of electrostatically spinning the composition at a location other than the skin to produce an adhesive fiber sheet which is composed of a fiber deposit and used via transfer onto the skin, and applying that adhesive fiber sheet to the skin. Such a method using an adhesive fiber sheet is a method which is different from the above-described electrostatic spraying step.

From the viewpoint of adhesive properties and the viewpoint of obtaining inconspicuous appearance, the thickness of the above-described adhesive fiber sheet is preferably set to 100 nm to 500 μm, more preferably 500 nm to 300 μm, further more preferably 1 μm to 100 μm, and even more preferably 10 μm to 50 μm, in terms of adhesive properties.

The color of the adhesive fiber sheet before transfer onto the skin is preferably transparent white or semi-transparent white. The L value thereof is preferably 80 or more, and more preferably 90 or more. Further, from the viewpoint of inconspicuous appearance, the a value and b value are preferably from −20 to 30, more preferably from −10 to 20, and further more preferably from 0 to 10. Note that the L value is the value defined in the CIE 1976 (L*, a*, b*) color space (CIELAB), where 100 is white and 0 is black.

It is preferable that the adhesive fiber sheet is produced by electrospinning on a peelable base material or formed on another base material by the electrospinning method and then laminated on a peelable base material. Here, as the peelable base material, it is possible to use non-woven sheets made of synthetic resins such as polyolefin resin or polyester resin, having a fiber diameter larger than that of the fiber sheet, or sheets with fine irregularities on the surface on which the fiber sheet of the coating is to be formed, for example, those with irregularities having height and width larger than the fiber thickness of the present invention but 1 mm or less, and more preferably 500 μm or less.

As described above, the present invention has been described based on its preferred embodiments, but the present invention is not limited to the above-described embodiments. For example, in the above-described embodiments, the person who wants to form a coating on his/her own skin grasps the electrostatic spraying device 10 and generates an electric field between the conductive nozzle of the device 10 and his/her skin, but as long as an electric field is generated between them, it is not necessary that the person who wants to form a coating on his/her own skin grasps the electrostatic spraying device 10.

With respect to the above-described embodiments, the present invention further discloses the following methods for producing a coating.

<1> A coating-forming composition for forming a coating composed of a fiber-containing deposit directly on the skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
   in which a mass content ratio of the component (B1) to the component (B), ((B1)/(B)), is 0.5 or more, and
   a mass content ratio of the component (B) to the component (C), ((B)/(C)), is 1 or more.

<2> A coating-forming composition for forming a coating composed of a fiber-containing deposit directly on the skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.

<3> A coating-forming composition for forming a coating composed of a fiber-containing deposit directly on the skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), (B)/(C), is 1 or more.

<4> A coating-forming composition for forming a coating composed of a fiber-containing deposit directly on the skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) is a polymer with a molecular weight distribution peak of more than 9.0×10⁴, and
   a mass content ratio of the component (B) to the component (C), (B)/(C), is 1 or more.

<5> The coating-forming composition according to any of the above <1> to <4>, in which the volatile substances of the component (A) preferably have a vapor pressure at 20° C. of 0.01 kPa or more and 106.66 kPa or less, more preferably 0.13 kPa or more and 66.66 kPa or less, further more preferably 0.67 kPa or more and 40.00 kPa or less, and even more preferably 1.33 kPa or more and 40.00 kPa or less.

<6> The coating-forming composition according to any of the above <1> to <5>, in which the volatile substances of the component (A) are alcohols, and the alcohols are preferably one or more selected from the group consisting of monovalent chain aliphatic alcohols, monovalent cyclic aliphatic alcohols, and monovalent aromatic alcohols, and the alcohols are more preferably one or more selected from the group consisting of ethanol, isopropyl alcohol, butyl alcohol, phenylethyl alcohol, propanol, and pentanol.

<7> The coating-forming composition according to any of the above <1> to <6>, in which the volatile substances of the component (A) are one or more selected from the group consisting of ethanol, isopropyl alcohol, butyl alcohol, and water, more preferably one or two selected from the group consisting of ethanol and butyl alcohol, and further more preferably those containing ethanol.

<8> The coating-forming composition according to any of the above <1> to <7>, in which the content of the component (A) in the composition is preferably 45% by mass or more, further more preferably 50% by mass or more, and even more preferably 55% by mass or more, and also preferably 95% by mass or less, further more preferably 94% by mass or less, and even more preferably 93% by mass or less, and the content of the component (A) in the composition is preferably 45% by mass or more and 95% by mass or less, further more preferably 50% by mass or more and 94% by mass or less, and even more preferably 55% by mass or more and 93% by mass or less.

<9> The coating-forming composition according to any of the above <1> to <8>, in which (B) the polymer having coating-forming ability used is preferably one or more selected from the group consisting of partially saponified polyvinyl alcohols, low saponified polyvinyl alcohols, completely saponified polyvinyl alcohols, polyvinyl butyral resins, polyurethane resins, polymethacrylic acid resins, oxazoline-modified silicones, polyvinyl acetal diethylaminoacetate, and polylactic acid, more preferably one or more selected from the group consisting of polyvinyl butyral resins, polylactic acid, and polyurethane resins, further more preferably contains a polyvinyl butyral resin, and particularly preferably contains a polyvinyl butyral resin as the main component.

<10> The coating-forming composition according to any of the above <1> and <5> to <9>, in which the mass content ratio of the component (B1) to the component (B), ((B1)/(B)), is preferably 0.5 or more, more preferably 0.51 or more, further more preferably 0.53 or more, and even more preferably 0.55 or more, and preferably 1 or less, more preferably 0.95 or less, and further more preferably 0.9 or less.

<11> The coating-forming composition according to any of the above <2> and <5> to <9>, in which the number average molecular weight of the component (B) is preferably 4.6×10⁴ or more, more preferably 4.7×10⁴ or more, and further more preferably 4.8×10⁴ or more, and also preferably 3×10⁵ or less, more preferably 2×10⁵ or less, and further more preferably 1.7×10⁵ or less.

<12> The coating-forming composition according to any of the above <3> and <5> to <9>, in which the number average molecular weight of the component (B) is preferably 4.6×10⁴ or more, more preferably 4.7×10⁴ or more, and further more preferably 4.8×10¹ or more, and preferably 3×10⁵ or less, more preferably 2×10⁵ or less, and further more preferably 1.7×10⁵ or less.

<13> The coating-forming composition according to any of the above <4> to <9>, in which the molecular weight distribution peak of the component (B) is preferably 9.5×10⁴ or more, more preferably 1.0×10⁵ or more, and further more preferably 1.1×10⁵ or more, and also preferably 1.0×10⁶ or less, more preferably 7.0×10⁵ or less, further more preferably 5.0×10⁵ or less, and even more preferably 3.5×10⁵ or less.

<14> The coating-forming composition according to any of the above <1> to <13>, in which the content of the component (B) is preferably 2% by mass or more, further more preferably 48 by mass or more, and even more preferably 5% by mass or more, also preferably 30% by mass or less, further more preferably 20% by mass or less, and even more preferably 15% by mass or less, and preferably 2% by mass or more and 30% by mass or less, further more preferably 4% by mass or more and 20% by mass or less, and even more preferably 5% by mass or more and 15% by mass or less.

<15> The coating-forming composition according to any of the above <1> to <14>, in which the mass content ratio of the component (B) to the component (A), ((B)/(A)), is preferably 0.03 or more and 0.5 or less, more preferably 0.04 or more and 0.4 or less, and further more preferably 0.055 or more and 0.3 or less.

<16> The coating-forming composition according to any of the above <1> to <15>, in which the polyols of the component (C) are one or more selected from the group consisting of alkylene glycols, polyalkylene glycols, and glycerins.

<17> The coating-forming composition according to any of the above <1> to <16>, in which the content of the component (C) is preferably 0.1% by mass or more, more preferably 0.58 by mass or more, and further more preferably 1% by mass, also preferably 308 by mass or less, more preferably 25% by mass or less, and further more preferably 20% by mass or less, and preferably 0.1% by mass or more and 30% by mass or less, more preferably 0.5% by mass or more and 25% by mass or less, and further more preferably 18 by mass or more and 20% by mass or less.

<18> The coating-forming composition according to any of the above <1> to <17>, in which the mass content ratio of the component (B) to the component (C), ((B)/(C)), is more preferably 1.1 or more and further more preferably 1.2 or more, also preferably 30 or less, more preferably 20 or less, and further more preferably 15 or less, and preferably 1 or more and 30 or less, more preferably 1.1 or more and 20 or less, and further more preferably 1.2 or more and 15 or less.

<19> The coating-forming composition according to any of the above <1> to <18>, further comprising a component (D) oil.

<20> The coating-forming composition according to the above <19>, in which the oil of the component (D) is preferably one or more selected from the group consisting of hydrocarbon oils, ester oils, higher alcohols, silicone oils, and fatty acids, and more preferably one or more selected from the group consisting of hydrocarbon oils, ester oils, and silicone oils.

<21> The coating-forming composition according to the above <19> or <20>, in which the content of the component (D) is preferably 0% by mass or more, more preferably 0.5% by mass or more, and further more preferably 1% by mass, also preferably 25% by mass or less, more preferably 20% by mass or less, and further more preferably 15% by mass or less, and preferably 0% by mass or more and 25% by mass or less, more preferably 0.5% by mass or more and 20% by mass or less, and further more preferably 18 by mass or more and 15% by mass or less.

<22> The coating-forming composition according to any of the above <1> to <21>, in which the viscosity at 25° C. is preferably 1 mPa·s or more and 5 000 mPa·s or less, further more preferably 10 mPa·s or more and 2 000 mPa·s or less, and even more preferably 50 mPa·s or more and 1 500 mPa·s or less.

<23> A method for producing a coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
   in which a mass content ratio of the component (B1) to the component (B), ((B1)/(B)), is 0.5 or more, and
   a mass content ratio of the component (B) to the component (C), ((B)/(C)), is 1 or more.

<24> A method for producing a coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.

<25> A method for producing a coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and a mass content ratio of the component (B) to the component (C), (B)/(C), is 1 or more.

<26> A method for producing a coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) is a polymer with a molecular weight distribution peak of more than 9.0×10⁴, and a mass content ratio of the component (B) to the component (C), (B)/(C), is 1 or more.

<27> The method for producing a coating according to any of the above <23> to <26>, wherein the object on which the coating is to be formed is skin.

<28> A method for applying a composition to an area which includes a site of a human where secretion of sebum is high, the composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
   in which a mass content ratio of the component (B1) to the component (B), ((B1)/(B)), is 0.5 or more, and
   a mass content ratio of the component (B) to the component (C), ((B)/(C)), is 1 or more.

<29> A method for applying a composition to an area which includes a site of a human where secretion of sebum is high, the composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.

<30> A method for applying a composition to an area which includes a site of a human where secretion of sebum is high, the composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and a mass content ratio of the component (B) to the component (C), (B)/(C), is 1 or more.

<31> A method for applying a composition to an area which includes a site of a human where secretion of sebum is high, the composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) is a polymer with a molecular weight distribution peak of more than 9.0×10⁴, and a mass content ratio of the component (B) to the component (C), (B)/(C), is 1 or more.

<32> A method for producing a sebum-resistant coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
   in which a mass content ratio of the component (B1) to the component (B), ((B1)/(B)), is 0.5 or more, and
   a mass content ratio of the component (B) to the component (C), ((B)/(C)), is 1 or more.

<33> A method for producing a sebum-resistant coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.

<34> A method for producing a sebum-resistant coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and a mass content ratio of the component (B) to the component (C), (B)/(C), is 1 or more.

<35> A method for producing a sebum-resistant coating, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) is a polymer with a molecular weight distribution peak of more than 9.0×10⁴, and a mass content ratio of the component (B) to the component (C), (B)/(C), is 1 or more.

<36> A method for applying a sebum-resistant sheet, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which (B1) a polymer with a molecular weight of 1×10⁵ or more is contained as the component (B), and
   in which a mass content ratio of the component (B1) to the component (B), ((B1)/(B)), is 0.5 or more, and
   a mass content ratio of the component (B) to the component (C), ((B)/(C)), is 1 or more.

<37> A method for applying a sebum-resistant sheet, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and
   a mass content ratio of the component (B) to the component (C), ((B)/(C)), is 1 or more.

<38> A method for applying a sebum-resistant sheet, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) has a number average molecular weight of 4.5×10⁴ or more, and a mass content ratio of the component (B) to the component (C), (B)/(C), is 1 or more.

<39> A method for applying a sebum-resistant sheet, comprising forming a coating composed of a fiber-containing deposit on the surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more selected from the group consisting of polyols,
   in which the component (B) is a polymer with a molecular weight distribution peak of more than 9.0×10⁴, and a mass content ratio of the component (B) to the component (C), (B)/(C), is 1 or more.

<40> An adhesive fiber sheet, which is composed of a fiber deposit and used via transfer onto the skin, the adhesive fiber sheet comprising: (B) a polyvinyl butyral resin as the main component of the fiber; and as a component (C), one or more selected from the group consisting of polyols, in which the component (B) has a number average molecular weight of 4.5×10⁴ or more, and the mass content ratio of the component (B) to the component (C), ((B)/(C)), is 1 or more.

<41> The adhesive fiber sheet according to the above <40>, in which the fiber constituting the adhesive fiber sheet has a thickness of 10 nm or more and 3 000 nm or less, preferably 50 nm or more and 1 500 nm or less, and more preferably 100 nm or more and 1 200 nm or less, in terms of equivalent circle diameter.

<42> The adhesive fiber sheet according to the above <40> or <41>, having a thickness of from 100 nm to 500 μm, preferably from 500 nm to 300 μm, more preferably from 1 μm to 100 μm, and further more preferably from 10 μm to 50 μm.

### EXAMPLES

Hereinafter, the present invention will be described in further detail with reference to Examples. However, the scope of the present invention is not restricted to such Examples. Unless otherwise noted, “%” means “% by mass”.

### Examples 1 to 11 and Comparative Examples 1 to 3

### (1) Preparation of Composition for Spraying

Compositions for spraying were prepared with the compositional features shown in Table 1 to Table 3. Note that the amount of ethanol shown in Tables 1 to 3 is the effective amount and does not include water.

### (2) Electrostatic Spraying Step

Using an electrostatic spraying device 10 having the configuration shown in FIG. 1 and the appearance shown in FIG. 2, the electrostatic spraying method was carried out directly toward the skin for 20 seconds. The conditions for the electrostatic spraying method were as follows.
Applied voltage: 10 kV
Distance between nozzle and skin: 100 mm
Amount of composition for spraying dispensed: 5 mL/h
Environment: 25° C., 30% RH

By this electrostatic spraying, a coating, in the form of a single film composed of a fiber deposit, was formed on the surface of the skin. The coating was a circle with a diameter of about 4 cm and a mass of about 3.8 mg. The thickness of the fiber measured by the above-described method was 660 nm.

### Evaluation

The coatings formed in Examples and Comparative Examples were evaluated for sebum resistance, texture, and octyl para-methoxycinnamate resistance by the following methods. The results are shown in Table 1 to Table 3.

### <Sebum Resistance>

Oleic acid was used as sebum. A 7 cm×7 cm coating was formed on aluminum foil of 5 cm×5 cm in the same manner as in the above-described electrostatic spraying step. Immediately after the coating formation, 1 μL of oleic acid was dropped onto the coating, and the time until a hole was formed in the coating was evaluated.

### Evaluation Criteria:

4: No hole formed for 6 hours.
3: No hole formed for 3 hours or longer.
2: No hole formed for 1 hour or longer.
1: Hole formed immediately after dropping oleic acid.

### <Texture (Coating Feeling/Stiffness/Discomfort)>

For the coatings formed with fibers by electrostatic spraying on the skin after application of skin care cosmetic (RISE lotion, Milk II) using Examples 1 to 11 and Comparative Examples 1 to 3, the coating feeling, stiffness, and discomfort were evaluated. As for the evaluation, the coating-forming composition was applied by electrostatic spraying to the skin immediately after the application of skin care cosmetic to form a coating formed with fibers, the coating was then lightly hand-pressed from above with the hand to completely adapt the coating, and after a lapse of 10 minutes, the coating feeling, stiffness, and discomfort were evaluated by sensory evaluation. The evaluation site was the cheek of the human face. Here, completely adapting the coating to the skin care cosmetics means that the coating is visually in a colorless and transparent state. The evaluation results are shown in Table 1. Further, the evaluation criteria were as follows.
5: Although the coating is attached to the skin during normal times, there is no particular coating feeling, stiffness, or discomfort, and even when the facial expression is changed, there is no coating feeling, stiffness, or discomfort, and the sensation is not different from the state without the coating.
4: Although the coating is attached to the skin during normal times, there is no particular coating feeling, stiffness, or discomfort, and when the facial expression is changed, there is slight coating feeling, stiffness, and discomfort, but it is possible to spend time without feeling any particular unpleasantness.
3: Although the coating is attached to the skin during normal times, there is no particular coating feeling, stiffness, or discomfort, but when the facial expression is changed, there is strong coating feeling, stiffness, and discomfort, and there is unpleasantness.
2: There is slight coating feeling, stiffness, and discomfort due to the coating attached to the skin during normal times, and especially when the facial expression is changed, there is strong coating feeling, stiffness, and discomfort, and there is unpleasantness.
1: There is strong coating feeling, stiffness, and discomfort due to the coating attached to the skin during normal times, and there is unpleasantness.

### <Octyl Para-Methoxycinnamate Resistance>

A 7 cm×7 cm coating was formed on aluminum foil of 5 cm×5 cm in the same manner as in the above-described electrostatic spraying step. On the coating, 1 μL of octyl para-methoxycinnamate/neopentyl glycol dicaprate=2/8 mixed solution was dropped onto the coating, and the time until a hole was formed in the coating was evaluated.

### Evaluation Criteria:

4: No hole formed.
3: No hole formed for 3 hours or longer.
2: No hole formed for 1 hour or longer.
1: Hole formed immediately after dropping.

### <Measurement of Molecular Weight and Distribution of Component (B)>

The molecular weight was determined by preparing a calibration curve from standard polystyrene using the gel permeation chromatography (GPC) method.
Device: HLC-8320 GPC (manufactured by Tosoh Corporation, detector integrated type)
Column: α−M+α−M (anion)
Eluent: 60 mmol/L phosphoric acid+50 mmol/L lithium bromide dimethylformamide solution
Flow rate: 1.0 mL/min
Column temperature: 40° C.
Detector: RI detector
Standard substance: polystyrene

TABLE 1

| | | Example | | | |
|---|---|---|---|---|---|
| | | 1 | 2 | 3 | 4 |
| (A) | Ethanol | 87.0 | 91.0 | 87.0 | 91.0 |
| (B) | Polyvinyl butyral (B60T manufactured by Kuraray Co., Ltd.) | 10.0 | | | |
| | Number average molecular weight 49000 | | | | |
| | Polyvinyl butyral (B75H manufactured by Kuraray Co., Ltd.) | | 6.0 | | |
| | Number average molecular weight 90000 | | | | |
| | Polyvinyl butyral (B60H manufactured by Kuraray Co., Ltd.) | | | 10.0 | |
| | Number average molecular weight 52000 | | | | |
| | Polyvinyl butyral (BX-5 manufactured by Sekisui Chemical Co., Ltd.) | | | | 6.0 |
| | Number average molecular weight 160000 | | | | |
| | Polyvinyl butyral (B60HH manufactured by Kuraray Co., Ltd.) | | | | |
| | Number average molecular weight 52000 | | | | |
| | Polyvinyl butyral (BM-1 manufactured by Sekisui Chemical Co., Ltd.) | | | | |
| | Number average molecular weight 40000 | | | | |
| (C) | PEG-8 (PEG-400 manufactured by Sanyo Chemical Industries, Ltd.) | 3.0 | 3.0 | 3.0 | 3.0 |
| | PPG-7 (ADEKA CARPOL DL-30 manufactured by Adeka Corporation) | | | | |
| | 1,3-Butylene glycol | | | | |
| (D) | Dimethylpolysiloxane 2cs | | | | |
| | (Silicone KF-96L-2cs manufactured by Shin-Etsu Chemical Co., Ltd.) | | | | |
| | Neopentyl glycol dicaprate | | | | |
| | (ESTEMOL N-01 manufactured by The Nisshin OilliO Group, Ltd.) | | | | |
| Total | | 100.0 | 100.0 | 100.0 | 100.0 |
| (A) total | | 87.0 | 91.0 | 87.0 | 91.0 |
| (B) total | | 10.0 | 6.0 | 10.0 | 6.0 |
| (C) total | | 3.0 | 3.0 | 3.0 | 3.0 |
| (D) total | | 0.0 | 0.0 | 0.0 | 0.0 |
| (B)/(C) | | 3.33 | 2.00 | 3.33 | 2.00 |
| (B) molecular weight corresponding to | | 115,000 | 242,000 | 121,000 | 339,000 |
| concentration fraction of 50% | | | | | |
| (B1)/(B) | | 0.55 | 0.78 | 0.57 | 0.87 |
| (B) molecular weight peak top | | 130538 | 316366 | 142994 | 392807 |
| Evaluation | Sebum resistance | >6 h | >6 h | 4 h | 1 h 10 m |
| | Sebum resistance score | 4 | 4 | 3 | 2 |
| | Discomfort feeling for A | 4 | 4 | 4 | 4 |
| | Discomfort feeling for P | 3 | 3 | 4 | 3 |
| | Discomfort feeling for N | 5 | 4 | 4 | 4 |
| | Evaluation of discomfort feeling, total score (N = 3) | 12 | 11 | 12 | 11 |

| | | Example | | |
|---|---|---|---|---|
| | | 5 | 6 | 7 |
| (A) | Ethanol | 87.0 | 87.0 | 91.0 |
| (B) | Polyvinyl butyral (B60T manufactured by Kuraray Co., Ltd.) | | 10.0 | |
| | Number average molecular weight 49000 | | | |
| | Polyvinyl butyral (B75H manufactured by Kuraray Co., Ltd.) | | | 6.0 |
| | Number average molecular weight 90000 | | | |
| | Polyvinyl butyral (B60H manufactured by Kuraray Co., Ltd.) | | | |
| | Number average molecular weight 52000 | | | |
| | Polyvinyl butyral (BX-5 manufactured by Sekisui Chemical Co., Ltd.) | | | |
| | Number average molecular weight 160000 | | | |
| | Polyvinyl butyral (B60HH manufactured by Kuraray Co., Ltd.) | 10.0 | | |
| | Number average molecular weight 52000 | | | |
| | Polyvinyl butyral (BM-1 manufactured by Sekisui Chemical Co., Ltd.) | | | |
| | Number average molecular weight 40000 | | | |
| (C) | PEG-8 (PEG-400 manufactured by Sanyo Chemical Industries, Ltd.) | 3.0 | | |
| | PPG-7 (ADEKA CARPOL DL-30 manufactured by Adeka Corporation) | | 3.0 | 3.0 |
| | 1,3-Butylene glycol | | | |
| (D) | Dimethylpolysiloxane 2cs | | | |
| | (Silicone KF-96L-2cs manufactured by Shin-Etsu Chemical Co., Ltd.) | | | |
| | Neopentyl glycol dicaprate | | | |
| | (ESTEMOL N-01 manufactured by The Nisshin OilliO Group, Ltd.) | | | |
| Total | | 100.0 | 100.0 | 100.0 |
| (A) total | | 87.0 | 87.0 | 91.0 |
| (B) total | | 10.0 | 10.0 | 6.0 |
| (C) total | | 3.0 | 3.0 | 3.0 |
| (D) total | | 0.0 | 0.0 | 0.0 |
| (B)/(C) | | 3.33 | 3.33 | 2.00 |
| (B) molecular weight corresponding to | | 118,000 | 115,000 | 242,000 |
| concentration fraction of 50% | | | | |
| (B1)/(B) | | 0.55 | 0.55 | 0.78 |
| (B) molecular weight peak top | | 114849 | 130538 | 316366 |
| Evaluation | Sebum resistance | 4 h | >6 h | >6 h |
| | Sebum resistance score | 3 | 4 | 4 |
| | Discomfort feeling for A | 4 | 4 | 4 |
| | Discomfort feeling for P | 5 | 4 | 4 |
| | Discomfort feeling for N | 5 | 5 | 5 |
| | Evaluation of discomfort feeling, total score (N = 3) | 14 | 13 | 13 |

TABLE 2

| | | Example | | | |
|---|---|---|---|---|---|
| | | 8 | 9 | 10 | 11 |
| (A) | Ethanol | 87.0 | 83.0 | 83.0 | 89.0 |
| (B) | Polyvinyl butyral (B60T manufactured by Kuraray Co., Ltd.) | 10.0 | 10.0 | 10.0 | |
| | Number average molecular weight 49000 | | | | |
| | Polyvinyl butyral (B75H manufactured by Kuraray Co., Ltd.) | | | | 4.0 |
| | Number average molecular weight 90000 | | | | |
| | Polyvinyl butyral (B60H manufactured by Kuraray Co., Ltd.) | | | | |
| | Number average molecular weight 52000 | | | | |
| | Polyvinyl butyral (BX-5 manufactured by Sekisui Chemical Co., Ltd.) | | | | |
| | Number average molecular weight 160000 | | | | |
| | Polyvinyl butyral (B60HH manufactured by Kuraray Co., Ltd.) | | | | |
| | Number average molecular weight 52000 | | | | |
| | Polyvinyl butyral (BM-1 manufactured by Sekisui Chemical Co., Ltd.) | | | | 4.0 |
| | Number average molecular weight 40000 | | | | |
| (C) | PEG-8 (PEG-400 manufactured by Sanyo Chemical Industries, Ltd.) | | 3.0 | 3.0 | 3.0 |
| | PPG-7 (ADEKA CARPOL DL-30 manufactured by Adeka Corporation) | | | | |
| | 1,3-Butylene glycol | 3.0 | | | |
| (D) | Dimethylpolysiloxane 2cs | | 4.0 | | |
| | (Silicone KF-96L-2cs manufactured by Shin-Etsu Chemical Co., Ltd.) | | | | |
| | Neopentyl glycol dicaprate | | | 4.0 | |
| | (ESTEMOL N-01 manufactured by The Nisshin OilliO Group, Ltd.) | | | | |
| Total | | 100.0 | 100.0 | 100.0 | 100.0 |
| (A) total | | 87.0 | 83.0 | 83.0 | 89.0 |
| (B) total | | 10.0 | 10.0 | 10.0 | 8.0 |
| (C) total | | 3.0 | 3.0 | 3.0 | 3.0 |
| (D) total | | 0.0 | 4.0 | 4.0 | 0.0 |
| (B)/(C) | | 3.33 | 3.33 | 3.33 | 2.67 |
| (B) molecular weight corresponding to | | 115,000 | 115,000 | 115,000 | 142,000 |
| concentration fraction of 50% | | | | | |
| (B1)/(B) | | 0.55 | 0.55 | 0.55 | 0.61 |
| (B) molecular weight peak top | | 130538 | 130538 | 130538 | 235152 |
| Evaluation | Sebum resistance | 4 h | 4 h | >6 h | >6 h |
| | Sebum resistance score | 3 | 3 | 4 | 4 |
| | Discomfort feeling for A | 4 | 4 | 4 | 4 |
| | Discomfort feeling for P | 5 | 4 | 3 | 3 |
| | Discomfort feeling for N | 5 | 5 | 4 | 5 |
| | Evaluation of discomfort feeling, total score (N = 3) | 14 | 13 | 11 | 12 |

| | | Comparative Example | | |
|---|---|---|---|---|
| | | 1 | 2 | 3 |
| (A) | Ethanol | 85.0 | 77.5 | 94.0 |
| (B) | Polyvinyl butyral (B60T manufactured by Kuraray Co., Ltd.) | | | |
| | Number average molecular weight 49000 | | | |
| | Polyvinyl butyral (B75H manufactured by Kuraray Co., Ltd.) | | | 6.0 |
| | Number average molecular weight 90000 | | | |
| | Polyvinyl butyral (B60H manufactured by Kuraray Co., Ltd.) | | 10.0 | |
| | Number average molecular weight 52000 | | | |
| | Polyvinyl butyral (BX-5 manufactured by Sekisui Chemical Co., Ltd.) | | | |
| | Number average molecular weight 160000 | | | |
| | Polyvinyl butyral (B60HH manufactured by Kuraray Co., Ltd.) | | | |
| | Number average molecular weight 52000 | | | |
| | Polyvinyl butyral (BM-1 manufactured by Sekisui Chemical Co., Ltd.) | 12.0 | | |
| | Number average molecular weight 40000 | | | |
| (C) | PEG-8 (PEG-400 manufactured by Sanyo Chemical Industries, Ltd.) | 3.0 | 12.5 | |
| | PPG-7 (ADEKA CARPOL DL-30 manufactured by Adeka Corporation) | | | |
| | 1,3-Butylene glycol | | | |
| (D) | Dimethylpolysiloxane 2cs | | | |
| | (Silicone KF-96L-2cs manufactured by Shin-Etsu Chemical Co., Ltd.) | | | |
| | Neopentyl glycol dicaprate | | | |
| | (ESTEMOL N-01 manufactured by The Nisshin OilliO Group, Ltd.) | | | |
| Total | | 100.0 | 100.0 | 100.0 |
| (A) total | | 85.0 | 77.5 | 94.0 |
| (B) total | | 12.0 | 10.0 | 6.0 |
| (C) total | | 3.0 | 12.5 | 0.0 |
| (D) total | | 0.0 | 0.0 | 0.0 |
| (B)/(C) | | — | 0.80 | — |
| (B) molecular weight corresponding to | | 84,000 | 121,000 | 242,000 |
| concentration fraction of 50% | | | | |
| (B1)/(B) | | 0.43 | 0.57 | 0.78 |
| (B) molecular weight peak top | | 85502 | 142994 | 316366 |
| Evaluation | Sebum resistance | 1 s | 1 s | >6 h |
| | Sebum resistance score | 1 | 1 | 4 |
| | Discomfort feeling for A | 5 | 5 | 2 |
| | Discomfort feeling for P | 4 | 5 | 2 |
| | Discomfort feeling for N | 5 | 5 | 2 |
| | Evaluation of discomfort feeling, total score (N = 3) | 14 | 15 | 6 |

TABLE 3

| | | Example | |
|---|---|---|---|
| | | 3 | 5 |
| (A) | Ethanol | 87.0 | 87.0 |
| (B) | Polyvinyl butyral | 10.0 | |
| | (B60H manufactured by Kuraray | | |
| | Co., Ltd) Number average molecular | | |
| | weight 52000, degree of | | |
| | acetalization 66.9 to 73.1, average 70 | | |
| | Polyvinyl butyral (B60HH manufactured | | 10.0 |
| | by Kuraray Co., Ltd.) Number average | | |
| | molecular weight 52000, degree of | | |
| | acetalization 73.3 to 81.2, average 77.25 | | |
| (C) | PEG-8 (PEG-400 manufactured by | 3.0 | 3.0 |
| | Sanyo Chemical Industries, Ltd.) | | |
| Total | | 100.0 | 100.0 |
| (A) total | | 87.0 | 87.0 |
| (B) total | | 10.0 | 10.0 |
| (C) total | | 3.0 | 3.0 |
| (B)/(C) | | 3.33 | 3.33 |
| (B1)/(B) | | 0.57 | 0.55 |
| (B) molecular weight peak top | | 142994 | 114849 |
| Evaluation | Octyl para-methoxycinnamate resistance | Not | 2 h |
| | | formed. | |
| | Octyl para-methoxycinnamate resistance | 4 | 2 |
| | score | | |

### <Evaluation of Peelability of Coating Containing Sebum>

4 μL of oleic acid was applied to artificial leather Sapurare of 5×5 cm² and 50 mg of emulsion (RISE MILK II) was applied on top of it. Furthermore, the compositions of Examples 2 and 3 and Comparative Examples 1 and 2 were electrostatically sprayed in the same manner as described above to form a coating composed of a fiber deposit with the same basis weight. After 15 minutes of standing, the coating was peeled off by grabbing the edge thereof, and the peelability of the coating containing sebum (oleic acid) was evaluated. The evaluation criteria for peelability were as follows. The results are shown in Table 4.

### Evaluation Criterion 1

“The state that the coating mixed with sebum is peeled off (the coating can be peeled off as one piece)”
4: The coating can be peeled off as a single clean film.
3: Small holes are formed, but the coating can be peeled off as a single film.
2: Large holes are formed, and the coating torn.
1: The film crumbles and cannot be peeled off.

### Evaluation Criterion 2

“Ease of peeling off the coating mixed with sebum”
4: The coating can be peeled off lightly and easily.
3: Some parts are difficult to peel off, but the coating can be peeled off upon application of force.
2: Many parts of the coating are difficult to peel off, and the coating can only be partially peeled off.
1: Most parts can hardly be peeled off.

TABLE 4

| | | | | Comparative | |
|---|---|---|---|---|---|
| | | Example | | Example | |
| | | 2 | 3 | 1 | 2 |
| (A) | Ethanol | 91.0 | 87.0 | 85.0 | 77.5 |
| (B) | Polyvinyl butyral | 6.0 | | | |
| | (B75H manufactured by Kuraray | | | | |
| | Co., Ltd.) Number average molecular | | | | |
| | weight 90000 | | | | |
| | Polyvinyl butyral | | 10.0 | | 10.0 |
| | (B60H manufactured by Kuraray | | | | |
| | Co., Ltd.) Number average molecular | | | | |
| | weight 52000 | | | | |
| | Polyvinyl butyral (BM-1 manufactured | | | 12.0 | |
| | by Sekisui Chemical Co., Ltd.) | | | | |
| | Number average molecular weight | | | | |
| | 40000 | | | | |
| (C) | PEG-8 (PEG-400 manufactuured by | 3.0 | 3.0 | 3.0 | 12.5 |
| | Sanyo Chemical Industries, Ltd.) | | | | |
| Total | | 100.0 | 100.0 | 100.0 | 100.0 |
| (A) total | | 91.0 | 87.0 | 85.0 | 77.5 |
| (B) total | | 6.0 | 10.0 | 12.0 | 10.0 |
| (C) total | | 3.0 | 3.0 | 3.0 | 12.5 |
| (B)/(C) | | 2.00 | 3.33 | — | 0.80 |
| (B1)/(B) | | 0.78 | 0.57 | 0.43 | 0.57 |
| (B) molecular weight peak top | | 316366 | 142994 | 85502 | 142994 |
| Evaluation | The way the coating mixed with sebum | 3 | 4 | 2 | 1 |
| | is peeled off (the coating can be peeled | | | | |
| | off as one piece) | | | | |
| | Ease of peeling off the coating | 3 | 4 | 3 | 1 |
| | mixed with sebum | | | | |

### REFERENCE SIGNS LIST

10 Electrostatic spraying device
11 Low voltage power source
12 High voltage power source
13 Auxiliary electrical circuit
14 Micro gear pump
15 Container
16 Nozzle
17 Conduit line
18 Flexible conduit line
19 Current-limiting resistor
20 Housing

1. A coating-forming composition for forming a coating composed of a fiber-containing deposit directly on skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more polyols,
wherein (B1) a polymer with a molecular weight of 1×10⁵ or more is contained as the component (B), and
wherein a mass content ratio of the component (B1) to the component (B), ((B1)/(B)), is 0.5 or more, and
a mass content ratio of the component (B) to the component (C), ((B)/(C)), is 1 or more.

2. A coating-forming composition for forming a coating composed of a fiber-containing deposit directly on skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more polyols,
wherein the component (B) has a number average molecular weight of 4.5×10⁴ or more, and
a mass content ratio of the component (B) to the component (C), ((B)/(C)), is 1 or more.

3. The coating-forming composition according to claim 1, wherein a content of the component (A) is 45% by mass or more and 95% by mass or less, and a content of the component (B) is 3% by mass or more and 30% by mass or less.

4. The coating-forming composition according to claim 1, wherein a content of the component (C) is 0.1% by mass or more and 30% by mass or less.

5. The coating-forming composition according to claim 1, wherein (B) the water-insoluble polymer having fiber-forming ability is at least one selected from the group consisting of partially saponified polyvinyl alcohols, low saponified polyvinyl alcohols, completely saponified polyvinyl alcohols, polyvinyl butyral resins, polyurethane resins, polymethacrylic acid resins, oxazoline-modified silicones, polyvinyl acetal diethylaminoacetate, and polylactic acid.

6. The coating-forming composition according to claim 1, wherein a mass content ratio of the component (B) to the component (C), ((B)/(C)), is 1.1 or more and 30 or less.

7. The coating-forming composition according to claim 1, wherein a mass content ratio of the component (B) to the component (A), ((B)/(A)), is 0.03 or more and 0.5 or less.

8. The coating-forming composition according to claim 1, further comprising an oil as a component (D).

9. A coating-forming composition for forming a coating composed of a fiber-containing deposit directly on skin via electrostatic spraying, obtained by adding the following component (A), component (B), and component (C):
(A) one or more volatile substances selected from the group consisting of water, alcohols, and ketones;
(B) a water-insoluble polymer having fiber-forming ability; and
(C) one or more polyols,
wherein the component (B) has a number average molecular weight of 4.5×10⁴ or more, and
a mass content ratio of the component (B) to the component (C), (B)/(C), is 1 or more.

10. A coating-forming composition for forming a coating composed of a fiber-containing deposit directly on skin via electrostatic spraying, the coating-forming composition comprising the following component (A), component (B), and component (C):
(A) one or more volatile substances selected from the group consisting of water, alcohols, and ketones;
(B) a water-insoluble polymer having fiber-forming ability; and
(C) one or more polyols,
wherein the component (B) is a polymer with a molecular weight distribution peak of more than 9.0×10⁴, and
a mass content ratio of the component (B) to the component (C), (B)/(C), is 1 or more.

11. A method for producing a coating, comprising forming a coating composed of a fiber-containing deposit on a surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more polyols,
wherein (B1) a polymer with a molecular weight of 1×10⁵ or more is contained as the component (B),
wherein a mass content ratio of the component (B1) to the component (B), ((B1)/(B)), is 0.5 or more, and
a mass content ratio of the component (B) to the component (C), ((B)/(C)), is 1 or more.

12. A method for producing a coating, comprising forming a coating composed of a fiber-containing deposit on a surface of an object on which the coating is to be formed, by using a composition comprising the following component (A), component (B), and component (C):
(A) one or more volatile substances selected from the group consisting of water, alcohols, and ketones;
(B) a water-insoluble polymer having fiber-forming ability; and
(C) one or more polyols,
wherein the component (B) has a number average molecular weight of 4.5×10⁴ or more, and
a mass content ratio of the component (B) to the component (C), ((B)/(C)), is 1 or more.

13. The method for producing a coating according to claim 11, wherein the object on which the coating is to be formed is skin.

14. An adhesive fiber sheet, which is composed of a fiber deposit and used via transfer onto skin, the adhesive fiber sheet comprising:
(B) a polyvinyl butyral resin as a main component of the fiber; and as a component (C), one or more polyols,
wherein the component (B) has a number average molecular weight of 4.5×10⁴ or more, and a mass content ratio of the component (B) to the component (C), ((B)/(C)), is 1 or more.

15. The adhesive fiber sheet according to claim 14, wherein the fiber constituting the adhesive fiber sheet has a thickness of 10 nm or more and 3 000 nm or less in terms of equivalent circle diameter.
